(19) United States
(12) Patent Application Publication
(10) Pub. No.: US 2008/0195214 A1
(43) Pub. Date: Aug. 14, 2008

(54) Co-Cr-Mo Alloy for Artificial Joint Having Excellent Wear Resistance

(76) Inventors: Akihiko Chiba, Iwate (JP); Kazushige Kumagai, Iwate (JP); Naoyuki Nomura, Iwate (JP)

Correspondence Address: HAYES SOLOWAY P.C., 3450 E. SUNRISE DRIVE, SUITE 140, TUCSON, AZ, 85718, US

(21) Appl. No.: 11/909,979

(22) PCT Filed: Mar. 28, 2005
(86) PCT No.: PCT/JP05/05785

§ 371 Date: Sep. 27, 2007

(51) Int. Cl. A61F 2/30; C22C 19/07; B22F 3/11; C22C 1/04

(52) U.S. Cl. 623/18.11; 420/436; 419/2; 419/23

(57) ABSTRACT

In the field of biocompatible Co—Cr—Mo alloys for use in artificial joints or the like, the formation of wear debris in a living body is a matter of concern. The purpose of the invention is to provide a technique for improving the wear resistance of a Co—Cr—Mo alloy for use in an artificial joint to thereby prevent the formation of wear debris in a living body. The improvement in wear resistance of a Co—Cr—Mo alloy for use in an artificial joint can be achieved by finely dividing the crystal particles of the alloy, by preparing an alloy composition having a higher Mo content than any known standard Co—Cr—Mo alloy (e.g., Co-29Cr-6Mo alloy), by increasing the proportion of the σ phase that is dispersedly precipitated, by sintering an alloy powder produced by gas atomization technique to form pores on the surface of the alloy material, or the like. The Co—Cr—Mo alloy having high wear resistance is applicable to medical devices including artificial hip joints, artificial knee joints and the like which have less biotoxicity, namely which are safer and have a longer useful life.

### TECHNICAL FIELD

The present invention relates to a Co—Cr—Mo alloy for artificial joints having excellent wear resistance, a method for manufacturing the same, and a biological material and artificial replacement material manufactured from this alloy. The present invention provides a technique for improving the wear resistance characteristics of Co—Cr—Mo alloys for use in artificial joints, and for suppressing the generation of wear debris in the living body.

### BACKGROUND ART

Co—Cr—Mo alloys are superior in terms of corrosion resistance and wear resistance. Because of this reliability, such alloys are used in parts that have sliding surfaces such as artificial hip joints and the like, prosthetic materials such as artificial bones, and various medical devices such as surgical implants and the like. In particular, since Co—Cr—Mo alloys are superior in terms of wear resistance characteristics, these alloys are used in artificial hip joints and the like. Conventionally, furthermore, artificial hip joints have commonly been constructed from a combination of a femoral epiphysis made of a Co—Cr—Mo alloy and a socket made of a high-density polyethylene (ultra-high molecular weight polyethylene: UHMWPE).

Recently, however, cases of bone resorption caused by UHMWPE wear debris have been reported, and the increased use of so-called metal-on-metal artificial hip joints in which both the epiphysis and socket are constructed from a Co—Cr—Mo alloy has been seen. Almost all of these metal-on-metal artificial hip joints use Co—Cr—Mo alloys used in casting (corresponding to F75 alloys in ASTM standards). In this case, the generation of wear debris of the Co—Cr—Mo alloy is a problem. The generation of wear debris in the living body is a problem that cannot be avoided as long as a casting Co—Cr—Mo alloy (corresponding to an F75 alloy in ASTM standards) is used in artificial hip joints. There is a demand for the development of a new material that can solve this problem.

### DISCLOSURE OF THE INVENTION

### Problems to Be Solved by the Invention

Accordingly, the present inventors conducted a wide-ranging search and performed diligent research in order to solve the abovementioned problem. As a result, the inventors confirmed the following facts: namely, conventional metal-on-metal artificial hip joint materials are almost all materials that use an ASTM standard F75 alloy containing approximately 0.3% carbon; furthermore, the reason for this is that these materials utilize a carbide phase that is deposited in a disperse manner as a mechanism for improving the wear resistance characteristics. Tn this case, however, the high-hardness carbide phase catches against the other material and causes wear (abrasive wear); accordingly, such materials are unsuitable for use in applications where the same type of material is used in the joint surfaces, as in metal-on-metal. Consequently, there is a need for the development of alloys for use in the living body which have high wear resistance characteristics that do not depend on carbide deposition reinforcement.

Thus, the present inventors succeeded in discovering that the wear resistance characteristics of Co—Cr—Mo alloys for use in artificial joints can be improved when the structure of such alloys is made finer by performing high-temperature working such as high-temperature forging or the like on an alloy composition, e.g., the F75 alloy composition of ASTM standards, but containing no carbon, e.g., a Co-29Cr-6Mo alloy, as an alloy for use in artificial joints, and that the generation of wear debris in the living body can be suppressed (an amount of wear that is 1/10 that seen in a conventional product can be realized). The inventors perfected the present invention on the basis of these discoveries. It was discovered that when the crystal grain size of a Co—Cr—Mo alloy for use in artificial joints (e.g., a Co-29Cr-6Mo alloy) is made finer, for example, when the crystal grain size is reduced to approximately 20 μm, the wear resistance characteristics can be greatly improved even if the alloy contains no carbide. Furthermore, in alloys in which the wear resistance characteristics are thus improved by reducing the crystal grain size, when a wear test was performed in quasi-biological fluids, it was also discovered that the wear resistance characteristics were greatly improved over those of the F75 alloy according to ASTM standards containing carbides as in conventional materials of the same type. This is a result that is attributable to the fact that there are no carbides that attack the other material.

Furthermore, the present inventors also discovered that when alloys containing more Mo than Co—Cr—Mo alloys for use in artificial joints (e.g., Co-29Cr-6Mo alloys), i.e., Co-29Cr-8Mo, Co-29Cr-10Mo, and the like are similarly caused to undergo a reduction in crystal grain size by high-temperature forging, such alloys with a reduced crystal grain size show wear resistance characteristics that are far better than those of conventional materials, and the inventors were also able to confirm that this appears to be due to the fact that the σ phase that is deposited in a disperse manner has an effect in improving the hardness of the alloy as a whole.

Furthermore, the present inventors also made it clear that in a sinter obtained by finely powdering a forged Co—Cr—Mo alloy for use in artificial joints (e.g., a Co-29Cr-6Mo alloy) by the gas atomization method, and then sintering this fine powder, if pores are appropriately formed in the surface of the material, a higher lubricating effect can be obtained, and such materials are extremely superior as medical materials, e.g., materials for use in artificial hip joints.

Thus, the present invention provides the following aspects:

The present invention provides a method for improving the wear resistance characteristics of Co—Cr—Mo alloys for use in artificial joints, characterized in that a treatment selected from the following group is performed: (1) a treatment that reduces the crystal grain size of the alloy, (2) adjustment of the alloy composition in which the Mo content is enriched, (3) a treatment that reinforces the dispersed deposition of the σ phase, (4) a powder sinter formation treatment, and (5) a treatment that forms pores generated in the powder sinter. In a desirable aspect, a Co—Cr—Mo cast alloy is subjected to a high-temperature forging treatment, and the crystal grain size of the alloy is reduced to a fine grain size. In a typical case, in the present invention, the mean grain size of the alloy crystals is set at a grain size selected from the group consisting of (1) 20 μm or less, (2) 15 μm or less, (3) 13 μm or less, (4) 11 μm or less, (5) 9 μm or less, (6) 7 μm or less, (7) 5 μm or less, (8) 4 μm or less, (9) 3.5 μm or less, (10) 3 μm or less, (11) 2.5 μm or less, (12) 2 μm or less, (13) 1.5 μm or less, and (14) 1 μm or less. Furthermore, the present invention provides a Co—Cr—Mo alloy for use in artificial joints superior in terms of wear resistance characteristics, which is characterized in that an alloy in which the Mo content is more enriched than in Co—Cr—Mo alloys according to universally known standards (e.g. corresponding to F75 of the ASTM standards, typically a Co-29Cr-6Mo alloy), and a method for manufacturing the same. More preferably, the Mo content of this alloy having a high wear resistance may be set at a content selected from the group consisting of (1) 6 mass % or greater, (2) 6.5 mass % or greater, (3) 7 mass % or greater, (4) 7.5 mass % or greater, (5) 8 mass % or greater, (6) 8.5 mass % or greater, (7) 9 mass % or greater, (8) 9.5 mass % or greater, (9) 10 mass % or greater, (10) 11 mass % or greater, and (11) 12 mass % or greater. The method of the present invention for improving the wear resistance characteristics of a Co—Cr—Mo alloy may include preparation of the alloy composition in which the Mo content is enriched to a content greater than that of a Co—Cr—Mo alloy according to a universally known standard (e.g., corresponding to F75 of the ASTM standards, typically a Co-29Cr-6Mo alloy), performing a high-temperature forging treatment, and reinforcing the dispersed deposition of the σ phase. Alloys that are the object of the present invention may be alloys in which the content of elements other than Co, Cr and Mo in the alloy composition is 1 mass % or less. Furthermore, the method of the present invention for improving the wear resistance characteristics of Co—Cr—Mo alloys may include subjecting the cast alloy to a gas atomization method, sintering the resulting alloy powder, and forming pores in the surface of the material. For example, the sintering of the alloy powder may be performed at a temperature selected from the group consisting of (1) 600° C. to 1350° C., (2) 650° C. to 1300° C., (3) 700° C. to 1250° C., (4) 750° C. to 1200° C., (5) 800° C. to 1150° C., (6) 850° C. to 1100° C., (7) 875° C. to 1060° C., and (8) 900° C. to 1050° C. Meanwhile, the sintering of the alloy powder may be performed at a pressure selected from the group consisting of (1) 10 to 250 MPa, (2) 20 to 200 MPa, (3) 25 to 150 MPa, (4) 30 to 150 MPa, (5) 30 to 100 MPa, (6) 30 to 80 MPa, (7) 35 to 50 MPa, (8) 35 to 45 MPa, and (9) 10 to 60 MPa.

Thus, the present invention provides a Co—Cr—Mo alloy for use in artificial joints in which the wear resistance characteristics are improved, characterized in that this alloy [is treated by a treatment] selected from the group consisting of [treatments in which] (1) the crystal grain size of the alloy is reduced to a fine grain size, (2) the alloy has a composition in which the Mo content is enriched, (3) the dispersed deposition of the σ phase is reinforced, (4) a powder sinter formation treatment is performed, and (5) pores are formed in the powder sinter. The Co—Cr—Mo alloy manufactured by the method of the present invention for improving wear resistance characteristics appears to be novel, and to have a sufficient inventive step. Medical devices, e.g., artificial joints and the like, which are characterized in that these medical devices are manufactured from this Co—Cr—Mo alloy having a high wear resistance, are provided by the present invention. These medical devices may include an epiphysis part and an acetabular roof for use in artificial joints (sockets for use in artificial joints), and the like.

### Effect of the Invention

A technique is provided in which the wear resistance characteristics of Co—Cr—Mo alloys for use in artificial joints is improved, and the generation of wear debris in the living body is conspicuously suppressed (an amount of wear that is 1/10 that seen in conventional products is realized). This technique can be applied to medical devices such as artificial hip joints, artificial knee joints, and the like, which have little toxicity in the living body, i.e., which are safer, and which have a long useful life. In the present invention, a method that does not depend on carbide reinforcement, i.e., a method in which the crystal grain size is reduced to a fine grain size, and/or a method in which the dispersed deposition of the σ phase is reinforced, is employed, and an increase in hardness is achieved. As a result, the present invention has superior points not seen in conventional techniques, i.e., the attack on the other materials involved, which is a problem in combinations of materials of the same type, can be suppressed. Consequently, the amount of wear debris generated in joint surfaces made of materials of the same type in artificial joints can be greatly reduced, the problem of relaxation of artificial joints can be solved, and the useful life can be greatly lengthened.

Other objects, characteristics, advantages, and aspects of the present invention will be apparent to one skilled in the art from the description given below. However, it should be understood that the following description and the description of the present specification, which includes specific examples and the like, merely show preferred modes of the present invention and are given only by way of explanation. It will be clearly apparent to one skilled in the art from the information given in the following description and other portions of the present specification that various changes and/or improvements (or modifications) are possible within the intention and scope of the present invention as disclosed in the present specification. All patent references and other references cited in the present specification are cited for descriptive purposes, and the contents of the references shall be construed as being included in the disclosure of the present specification as part of the present specification.

### BRIEF DESCRIPTION OF THE DRAWINGS

FIG. 1 shows a pin-on-disk wear test apparatus used in order to evaluate the wear resistance of the manufactured alloys;

FIG. 2 shows the optical-microscopic structure (photograph) of ASTM F75 (a), and a Co-29Cr-6Mo alloy manufactured using high-temperature forging (in which the crystal grain size was reduced to a fine grain size) [(b) mean grain size 14 μm, (c) mean grain size 3 μm];

FIG. 3 shows the wear test results for ASTM F75 and a Co-29Cr-6Mo alloy manufactured using high-temperature forging (in which the crystal grain size was reduced to a fine size);

FIG. 4 shows the optical-microscopic structures (photographs) of Co-29Cr-xMo (x=6, 8, 10) forged alloys in which the amount of Mo added was increased to 6, 8, and 10 mass %, with (a) showing a Co-29Cr-6Mo alloy, (b) showing a Co-29Cr-8Mo alloy, and (c) showing a Co-29Cr-10Mo alloy;

FIG. 5 shows the results of the wear test for Co-29Cr-xMo (x=6, 8, 10) forged alloys in which the amount of Mo added was increased to 6, 8, and 10 mass %, and for ASTM F75;

FIG. 6 shows the optical-microscopic structures of sinters obtained by sintering an atomized alloy powder from a Co-29Cr-6Mo cast alloy, with the left side showing a sinter obtained at a pressing pressure of 40 MPa and a temperature of 936° C., and the right side showing a sinter obtained at a pressing pressure of 40 MPa and a temperature of 1052° C.; and

FIG. 7 shows the results of the wear test for a 936° C. sinter and a 1052° C. sinter obtained by sintering an atomized alloy powder from a Co-29Cr-6Mo cast material, with the wear test results also being shown for ASTM F75 and a Co-29Cr-6Mo cast material (crystal grain size 12 μm).

### BEST MODE FOR CARRYING OUT THE INVENTION

In the present invention, the term “Co—Cr—Mo alloy” refers to an alloy using Co as a base, and containing substantial proportions of chromium (Cr) and molybdenum (Mo); examples include alloys included in the group known in the field as “super alloys”. The term “super alloy” is a technical term generally used to refer to alloys having an extremely high strength, and superior mechanical characteristics and wear resistance; it is recognized that typical super alloys have a stable micro-structure. These Co—Cr—Mo alloys have a superior biocompatibility, a high yield strength, superior hardness, and the like. Examples of such Co—Cr—Mo alloys may be cited from ASTM (American Society for Testing and Materials) standards, e.g., ASTM F1537 94, ASTM F799, ASTM F75, and the like, and ISO (International Organization for Standardization) standards, e.g., ISO 5832-12, and the like.

The alloy composition (wt %) according to the ASTM F1537 94 standard is as follows:

Mo: 5.0 to 7.0 wt %, Cr: 26.0 to 30.0 wt %, C: ≦0.35 wt %, Ni: ≦1.0 wt %, Fe: ≦0.75 wt %, Mn: ≦1.0 wt %, Si: ≦1.0 wt %, N₂: ≦0.25 wt %, remainder Co.

Here, the Ni arises from unavoidable admixture in the raw material, and is ordinarily contained at the rate of approximately 0.2 to 1.0 wt %, and the Co constituting the remainder refers to the amount of Co excluding the impurity that occurs in trace amounts.

In regard to such Co—Cr—Mo alloys, Vitallium (commercial name) is known as a product for use in plastic surgery; the general composition of this alloy is as follows:

Mo: approximately 5.50 wt %, Cr: approximately 28.00 wt %, C: approximately 0.25 wt %, Mn: approximately 0.70 wt %, Si: approximately 0.75 wt %, remainder Co.

Here, the Ni arises from unavoidable admixture in the raw material, and is ordinarily contained at the rate of approximately 0.002 to 2.5 wt %, and the Co constituting the remainder refers to the amount of Co excluding the impurity that occurs in trace amounts.

Numerous examples of such Co—Cr—Mo alloys have been reported; for instance, these alloys may include the alloys disclosed in Japanese Laid-open Patent Application No. 2002-363675 (JP, A, 2002-363675), pamphlet of International Disclosure No. 97/00978 (WO, A, 97/00978), specification of U.S. Pat. No. 5,462,575 (U.S. Pat. No. 5,462,575), specification of U.S. Pat. No. 4,668,290 (U.S. Pat. No. 4,668,290), and the like, alloys obtained by modifying these alloys, alloys derived from these alloys, and the like. For example, as is disclosed in Japanese Laid-open Patent Application No. 2002-363675, alloys in which the amount of Mo is increased to an approximately value≦12.0 wt %, and alloys in which this amount is increased to approximately 10 wt %, may also be included.

In one concrete aspect, the Co—Cr—Mo alloy may have the following composition: Me: approximately 5.0 to 6.0 wt %, preferably 5.0 to 5.5 wt %, and more preferably 5.5 wt %, Cr: approximately 26.0 to 29.5 wt %, preferably 27.0 to 29.0 wt %, and more preferably 29.0 wt %, C: ≦approximately 0.35 wt %, preferably ≦approximately 0.07 wt %, Ni: ≦approximately 1.0 wt %, Fe: ≦approximately 1.5 wt %, preferably ≦approximately 0.75 wt %, Mn: ≦approximately 1.0 wt %, Si: ≦approximately 1.0 wt %, preferably ≦approximately 0.4 wt %, N₂: ≦0.25 wt %, and the remainder Co (here, the Ni arises from unavoidable admixture with the raw material, and is present in a content of at least approximately 0.002 wt %, or at the very lowest, in an amount greater than the order of 50 ppm, and the Co constituting the remainder is the amount of Co excluding the impurity that occurs in trace amounts). C, Fe, Si, N₂, and other trace elements may be unavoidably contained in the alloy.

In the ASTM F75 alloy seen in the past, a maximum of 0.35% carbon has been included for the purpose of forming carbides,

In the present invention, an increased amount of the element Mo is added to a raw material giving a composition that forms the abovementioned Co—Cr—Mo alloy (especially the alloy corresponding to F75 of the ASTM standard), and the alloy-producing composition thus obtained can be subjected to an ordinary alloy preparation method.

The mixture amount of the added element in the alloy composition can be increased or decreased so that a reinforcement of the dispersed deposition of the desired σ phase is obtained, and this mixture amount can be set in a range which is such that there is substantially no deleterious effect on the characteristics of the alloy obtained. For example, [the added element] can be added so that the content is a content selected from the group consisting of (1) 6 mass % or greater, (2) 6.5 mass % or greater, (3) 7 mass % or greater, (4) 7.5 mass % or greater, (5) 8 mass % or greater, (6) 8.5 mass % or greater, (7) 9 mass % or greater, (8) 9.5 mass % or greater, (9) 10 mass % or greater, (10) 11 mass % or greater, and (11) 12 mass % or greater. However, the present invention is not limited to this; the mixture amount can be varied in a range which is such that the desired object is achieved, and such that there is substantially no deleterious effect on the characteristics of the alloy obtained.

It appears that an effect which causes the crystal grain size to be reduced to a fine grain size, and which causes the σ phase to be deposited as a fine phase, is obtained as the amount of Mo is increased. The wear resistance characteristics of the alloy can thus be improved. In other words, as the amount of Me added is increased, a result is obtained which makes it possible to reduce the wear rate of the Co—Cr—Mo alloy.

In the present invention, raw materials with an ordinary alloy composition (especially raw materials with an alloy composition according to a universally known standard) or Mo-enriched alloy raw materials are put together, and, after being mixed if necessary, are heated and melted to produce a molten alloy. Besides vacuum induction melting (VIM), various universally known melting methods can be used. During the melting treatment step, a partial pressure of an inert gas such as argon gas or the like can be applied to the VIM furnace. As another method, an inert gas or a covering gas containing nitrogen gas can be caused to flow through the VIM furnace. In the presence of this inert gas or covering gas, the melted alloy is appropriately heated to a specified temperature at which a specified composition is obtained, or is held at a specified temperature. Next, the melted alloy can be cast into an ingot or body having a specified shape, and may be cooled without further processing. If necessary, the alloy can be quenched. Examples of quenching methods that can be used include water quenching, quenching with ice water, oil quenching, hot bath quenching, salt bath quenching, electrolytic quenching, vacuum quenching, air quenching, jet quenching, spray quenching, multi-stage quenching, time quenching, press quenching, partial quenching, forge quenching, and the like. These methods are used as appropriate. In a typical case, water quenching or quenching with ice water is used. The ingot can be worked into a desired shape by performing hot extrusion, hot rolling, hot drawing, or the like.

Furthermore, the alloy melt can be formed into an appropriate shape such as a thin band, fine wire, or the like by rapid cooling of the melt. A liquid spinning method, rotating liquid spinning method, Kavesh method, twin roll method, single roll method, and the like are among methods that can be used for the rapid cooling of the melt. Generally, in rapid cooling of the melt, the molten metal is discharged as a jet onto a cooled metal roll or into a coolant fluid, and solidified. This cooled metal roll is ordinarily caused to rotate at a high speed. Various types of fluids can be used as the abovementioned coolant fluid; there are no restrictions as long as a desirable result is obtained. For example, fluids containing silicone oils can be used. For instance, examples include polydimethylsiloxanes TSF451-30 and TSF440 manufactured by Toshiba Silicone Co.; however, the fluids used are not limited to these. Furthermore, these silicone oils may be used singly, or may be used in combinations of a plurality of oils. Furthermore, in order to remove gases such as low-boiling-point solvents. Dissolved air, and the like contained in ordinary silicone oils, there may be cases in which it is desirable to remove these gases by heating the silicone oil used beforehand under reduced pressure. Furthermore, in order to manufacture a fine metal wire directly by rapidly solidifying the molten metal in a silicone oil, it is desirable to suppress to a minimum any disturbance that is applied to the molten metal jet stream. For this purpose, it is desirable to achieve a fine balance between the molten metal jet and the silicone oil. In concrete terms, it is desirable to control velocity differences, differences in viscosity, differences in surface tension, and the like between the molten metal jet and the silicone oil. In particular, it is effective in the present invention to stipulate the viscosity of the silicone oil.

The rotating liquid spinning method refers to a method in which a fine metal wire is manufactured by forming a liquid layer via centrifugal force on the inside of a rotating drum, causing the molten metal or molten alloy to jet from a nozzle opening, and solidifying this metal or alloy in the liquid layer. For example, this is a technique in which water is used as a coolant, and a fine metal wire is obtained by causing the alloy to jet into the rotating water coolant in a molten state. For example, the Kavesh method is a method described in Japanese Laid-open Patent Application No. 49-135820 (JP, A, 49-135820 (Dec. 27, 1974)), and is a technique in which the melt is extruded into the form of a molten filament, and is caused to pass through a liquid-form rapid cooling region via a controlled gas-form interface region, and the filament and liquid-form coolant are caused to flow side by side in this liquid-form rapid cooling region. The coolant used here is a fluid coolant, and may be a pure liquid, solution, emulsion, or solid-liquid dispersion. The fluid medium may react with the melt to form a stable surface skin, or may be chemically non-reactive with the jetting melt. Furthermore, in selecting the rapid cooling medium, this selection is made in relation to the thermal capacity of jetting melt, and it is considered desirable to select a rapid cooling fluid which is colder, and/or which has a higher specific heat, density, heat of evaporation, and thermal conductivity, as the thermal capacity of the jetting melt increases. Furthermore, other desirable properties of the fluid rapid cooling medium are generally a low viscosity which minimizes splitting of the jetting melt, non-tackiness, non-toxicity, optical transparency, and low cost. In actuality, fluids such as water, a 23 wt % aqueous solution of sodium hydroxide at −20° C., a 21.6 wt % aqueous solution of magnesium chloride at −33° C., and a 51 wt % aqueous solution of zinc chloride at −62° C. are respectively desirable; furthermore, a silicone rapid cooling fluid such as Dow Corning 510 fluid with a viscosity of 50 centistokes at 0 to 100° C. or the like can also be used.

Furthermore, the cooled alloy can be appropriately worked. For example, a thin band, fine wire, or the like obtained by rapid cooling of the melt or the like can be subjected to shaping or the like if necessary, and can be formed into a medical device. In order to eliminate segregation and the like, the alloy can be further subjected to a homogenizing heat treatment. The homogenizing heat treatment may be a treatment comprising a heat treatment and a quenching treatment. The heat treatment can be performed using a method selected from methods that are universally known in the field; for example, this heat treatment can be performed using an electric furnace or the like. In a typical case, the alloy can be heated under reduced pressure or in a vacuum. Typically, for example, the alloy is heated for 5 to 30 hours, preferably 8 to 24 hours, and even more preferably 10 to 20 hours. In one concrete example, the alloy is heated for 12 to 15 hours. The heating temperature, for example, is 1400° C. or less, typically 900 to 1350° C., preferably 1000 to 1300° C., and even more preferably 1050 to 1250° C. However, as long as the desired object is achieved, the heating temperature is not limited to these temperatures. In one concrete example, the heating temperature is 1100 to 1200° C. In the homogenizing heat treatment, quenching can be performed following the abovementioned heat treatment. The quenching method used is the same as described above.

In the Co—Cr—Mo alloy of the present invention, an alloy in which internal defects are eliminated can be obtained by adjusting the thermal history. This thermal history adjustment treatment is one in which shrinkage cavities, gas bubbles, and the like occurring in forged alloys are crushed by forging, dendritic structures are also destroyed, and a uniform structure is formed by subsequent recrystallization annealing. In the adjustment of the structure, it may be expected that the growth of deposits can be suppressed by rapid-cooling casting using a water-cooled copper casting mold. The effect of rapid cooling during casting in suppressing the growth of deposits is conspicuous in cases where cooling is performed at a cooling rate of 1000° C./min or greater in the temperature range extending from the casting temperature to 400° C. The forged structure is destroyed by high-temperature casting, and a matrix comprising isoaxial crystal grains reduced to a size of 40 μm or less is formed. The reduction in the [crystal grain] size of the matrix is also effective in improving the wear resistance.

In the present invention, it is also possible to reduce the crystal grain size of the alloy, and to assist or reinforce the dispersed deposition of the σ phase, by selecting the heat treatment method and working temperature. In concrete terms, the high-temperature forging temperature can be set in the range of 1100 to 1400° C. in the system of the present invention. The forming of a fine dispersion in a matrix with the fine crystal grain size maintained can also be achieved by using rapid cooling such as water cooling or the like when the alloy obtained by high-temperature forging is brought down to room temperature.

In the present invention, the cast alloy can be subjected to a treatment that reduces the crystal grain size of the alloy to a fine grain size. By doing this, it is possible to achieve an improvement in the wear resistance characteristics. Typically, a reduction in the crystal grain size of the alloy can be achieved by subjecting the cast alloy to a high-temperature forging treatment. In this forging treatment, a treatment may be included in which the metal ingot is struck (wrought) in a high-temperature state, and may include a treatment that presses bubbles or gas (pores) contained in the metal ingot. Typically, a treatment that reduces the size of the crystal grains is included. This forging treatment may be a treatment that applies a compressive weight, or may be a treatment that includes mechanical forging or free forging. This treatment may be a treatment in which a force is applied between upper and lower anvils or the like using a press or hammer with the metal material in a high-temperature state, and may include drawing/swaging, hole boring, hole opening, expanding, necking, and the like, or combinations of these treatments. As appropriate, the forging treatment may be die forging, closed forging, forging using a hammer, press forging, or the like, and can be performed using a forging machine. Forging can be performed using a drop hammer, spring hammer, counter-blow hammer, air hammer, steam hammer, or a press such as a hydraulic press (oil-pressure press), knuckle joint press, friction press, or the like. It is desirable that the die used in die forging be heated beforehand. This is desirable since there is no loss of heat if this is done. This high-temperature forging can reduce the mean grain size of the alloy crystals to 40 μm or less. In some cases, the grain size can be further reduced to 30 μm or less, reduced to 20 μm or less, reduced to 15 μm or less, preferably reduced to 13 μm or less, reduced to 11 μm or less, reduced to 9 μm or less, or reduced to 7 μm or less. Forging can be performed until this is achieved. In the present invention, this high-temperature forging can further reduce the mean grain size of the alloy crystals to 5 μm or less, 4 μm or less, 3.5 μm or less, 3 μm or less, 2.5 μm or less, 2 82 m or less, 1.5 μm or less, or 1 μm or less. Forging can he performed until this is achieved. This forging can be performed until the desired wear resistance characteristics are obtained. The high-temperature forging can be performed at a temperature of 1000 to 1300° C., and can ideally be performed at a temperature of 1000 to 1200° C. However, the forging is not limited to this temperature range; for example, the forging can be performed at a temperature of 600° C. to 1350° C., and in some cases can be performed at a temperature of 650° C. to 1300° C., a temperature of 700° C. to 1250° C., a temperature of 750° C. to 1200° C., a temperature of 800° C. to 1150° C., a temperature of 850° C. to 1100° C., a temperature of 875° C. to 1060° C., or a temperature of 900° C. to 1050° C. Conditions at which the desired wear resistance characteristics are obtained may be appropriately selected. The weight applied during forging can be adjusted by varying the weight of the hammer in cases where a forging machine is used, and can be appropriately selected so that the desired wear resistance characteristics are obtained. For example, the striking of the material heated to the abovementioned temperature can be initiated using a 1- to 3-ton hammer, typically a 1.5-ton hammer, and striking with this hammer can be performed until the material temperature drops to the desired temperature or less. If necessary, forging can be performed so that the material is subsequently re-heated, and striking is re-initiated after the material has reached the desired temperature. The forged alloy can also be subjected to cold rolling, mechanical working, or the like.

The alloy of the present invention can be subjected to gas atomization of the metal as disclosed in Japanese Laid-Open Patent Application No. 62-80245 (JP, A, 62-80245 (Apr. 13, 1987)), or the technique disclosed in Japanese Laid-Open Patent Application No. 5-1345 (JP, A, 5-1345 (Jan. 8, 1993)) utilizing the mechanical alloying disclosed in the specification of U.S. Pat. No. 3,591,362 (U.S. Pat. No. 3,591,362) can be employed, and the alloy can be formed into an alloy having a configuration that is suitable for medical devices. For example, the alloy of the present invention containing an increased amount of Mo (alloy having an enriched Mo content) can be converted into a powder by the gas atomization method, the powder thus obtained can be compressed by a thermal mechanical treatment to form a solid alloy (sinter), and if necessary, an artificial replacement material can be manufactured by performing a working treatment such as a forging treatment or the like.

In the sintering treatment, for example, the atomized alloy powder can be screened to a size of 1 to 50 mesh (e.g., screened to a size of 10 mesh), placed in a soft steel vessel having an internal diameter of 5.08 cm (2 inches) or 7.62 cm (3 inches) and a height of 10.16 cm (4 inches), degassed by an ordinary method after the vessel is filled, and then heated to a temperature in the range of 600° C. to 1350° C. and subjected to a uniform pressure in the range of 10 to 250 MPa, and heated to a high temperature. Subsequently, the sinter product is cooled to the ambient temperature along with the vessel.

The particle size of the alloy powder can be appropriately selected in accordance with the object; for example, a powder having a mean particle size of 25 μm or greater can be suitably used. If the number of pores is to be reduced, it is desirable to use fine particles having a particle size of 25 μm or less; in order to heighten the lubricating effect by increasing the number of pores, for example, bead-form particles having a diameter of 200 to 600 μm can be advantageously used. For example, the sintering of the alloy powder can be performed at a temperature of can be performed at a temperature in the range of 600° C. to 1350° C., a temperature in the range of 650° C. to 1300° C., a temperature in the range of 700° C. to 1250° C., a temperature in the range of 750° C. to 1200° C., a temperature in the range of 800° C. to 1150° C., a temperature in the range of 850° C. to 1100° C., a temperature in the range of 875° C. to 1060° C., a temperature in the range of 900° C. to 1050° C., or the like. Ideally, a temperature in the range of 900° C. to 1250° C. may be cited as an example. Furthermore, the sintering of the alloy powder can be performed under a pressure in the range of 10 to 250 MPa, a pressure in the range of 20 to 200 MPa, a pressure in the range of 25 to 150 MPa, a pressure in the range of 30 to 150 MPa, a pressure in the range of 30 to 100 MPa, a pressure in the range of 30 to 80 MPa, a pressure in the range of 35 to 50 MPa, or a pressure in the range of 35 to 45 MPa. Ideally, a pressure in the range of 10 to 60 MPa or the like may be cited. Conditions under which the desired surface porosity can be obtained, or under which lubricating properties can be obtained, may be appropriately selected.

Examples of thermal mechanical treatments include the treatments described above; such treatments may include hot drawing, hot rolling, hot pressing, and the like. The product can then be mechanically worked, and finished to a smooth surface. If necessary, furthermore, the smooth surface can be treated, and a porous coating can also be formed.

Materials for use in the body and medical devices such as artificial replacement materials or the like can be manufactured from the highly wear-resistant Co—Cr—Mo alloy of the present invention, or from the Co—Cr—Mo alloy having high lubricating properties. Examples of medical devices include dental materials such as bridges, tooth roots, and the like, replacement materials such as artificial bone materials, surgical implants and the like, biocompatible implants, joint implants, medical artificial implants, and the like. Examples of implants and the like include artificial hips, artificial knees, artificial shoulders, artificial ankles, artificial elbows, other artificial joint implants, and the like. The alloy of the present invention can also be used to manufacture members for fixing bone fracture sites. Examples of such members may include pegs, screw pegs, nuts, screws, plates, pins, hook pins, hooks, fittings, embedding bases, and the like. Typical examples of such products include artificial joints for medical use such as artificial hip joints and the like. Members and products that have contact parts for medical members that can move relative to each other are also included; for example, such products include a bone epiphysis part and an acetabular roof for artificial joints (sockets for use in artificial joints), and the like.

The present invention accordingly provides a Co—Cr—Mo alloy for biological use which is used in artificial hip joints and the like, and a problem-solving technique with the object of solving the problem of the generation of wear debris in the living body. Accordingly, a technique is provided which improves the wear resistance characteristics of the Co—Cr—Mo alloy for use in artificial joints, and which suppresses the generation of wear debris in the living body. The improvement of the wear resistance characteristics of the Co—Cr—Mo alloy of the present invention for use in artificial joints is achieved by procedures such as reducing the size of the crystal grains of the alloy to a fine size, forming an alloy composition which contains more Mo than a Co—Cr—Mo alloy of a universally known standard (e.g., a Co-29Cr-6Mo alloy), heightening the proportion of the dispersed deposition of the σ phase, sintering an alloy powder produced by the gas atomization method, and forming pores in the surface of the alloy material, and the like. This Co—Cr—Mo alloy having a high wear resistance is suitable for use in medical devices such as artificial hip joints, artificial knee joints, and the like which have a low toxicity in the living body, i.e., which are safer, and which have a long service life.

In the present specification, superior wear resistance, reduction of the crystal grain size to a fine grain size, enrichment of the Mo content, reinforcement of the dispersed deposition of the σ phase, and a greater presence of pores in the surface of the material refers to these properties in comparison with an alloy corresponding to F75 of the ASTM standards, typically a Co-29Cr-6Mo alloy, e.g., a cast product.

### EXAMPLES

The present invention will be described in concrete terms below by giving examples. However, these examples are merely for the purpose of describing the present invention, and are given in order to provide a reference to concrete aspects. These examples are used to describe specified concrete aspects of the present invention, and do not restrict or limit the claims of the invention disclosed in the present application. In the present invention, it will be understood that various working configurations based on the concept of the present specification are possible.

All of the examples, other than examples described in detail otherwise, are examples that are or can be implemented using standard techniques, and are customary examples that will be understood by a person skilled in the art.

(Material Composition)

The nominal composition of the Co—Cr—Mo alloy in the examples is Co: bal., Cr: 29 mass %, Mo: 6, 8, 10 mass %.

(Manufacture of Test Specimens)

Ingots prepared using high-temperature forging were worked into disks having a diameter of 30 mm and a thickness of 5 mm using a wire-cutting discharge working machine. The forging was performed by striking the material heated to a temperature of approximately 1000 to 1200° C. with a 1.5-ton hammer; when the temperature dropped, the material was re-heated, and forging was performed at the abovementioned temperature. The operation was performed until the desired crystal grain size was obtained. The material worked into these disks was used as wear test specimens. After being emery polished, the wear test specimens were buffer-polished using 0.06-μm abrasive grains, and the surfaces of the specimens were finished to a calculated mean roughness Ra of 0.05 μm or less.

(Wear Test Method)

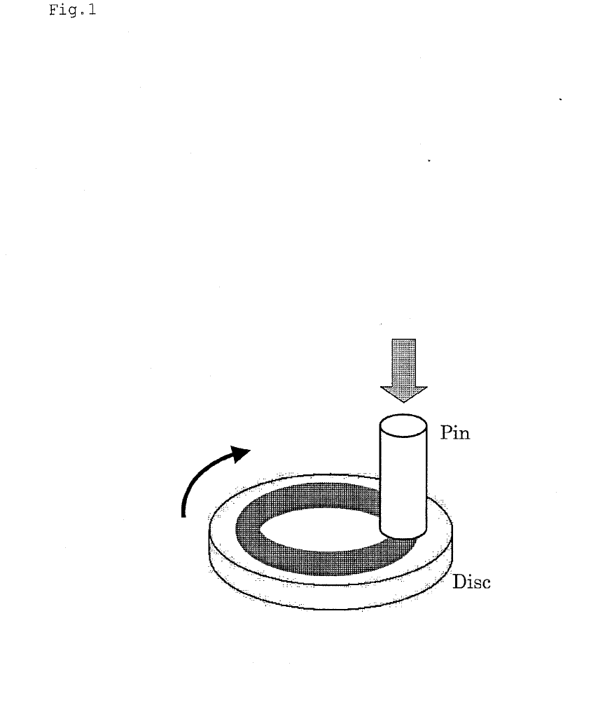

A pin-on-disk wear test (see FIG. 1) was performed in order to evaluate the wear resistance of the prepared alloy. The pin and disk used in this wear test were both made of the same material.

The test conditions were as shown below.

Test solution: Hanks' solution (inorganic quasi-biological liquid)

Weight: 9.8 N

Slipping distance: 1.21×10⁷ mm

Slipping speed: 20 mm/s

Temperature: 37±2° C.

The wear test was performed after the test specimens were cleaned by ultrasound in acetone.

(Evaluation of Wear Rate)

Following the completion of the test, the test specimen was removed and cleaned by ultrasound in acetone; then, the weight of the test specimen was measured, and the variation in the weight of the test specimen before and after the test was investigated.

The wear rate was calculated using the following equation on the basis of the variation in the weight thus obtained.

ω=Mₗₒₛₛ/(L×ρ)

Here, ω is the wear rate (amount of wear per unit slipping distance) [mm²], Mₗₒₛₛ is the variation in the weight of the test specimen [g], L is the slipping distance [mm], and ρ is the density of the test specimen [g/mm³].

(Test Results)

### Example 1

(Wear Test Results for Alloys With Reduced Crystal Grain Size)

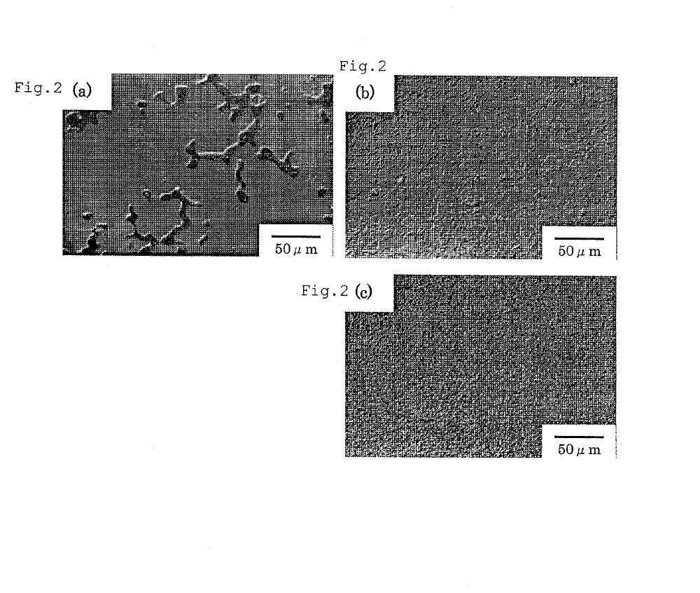

FIG. 2 shows the optical-microscopic structure of ASTM F75 (a), and of Co-29Cr-6Mo alloys (having a reduced crystal grain size) prepared by high-temperature forging, i.e., (b) mean grain size 14 μm, and (c) mean grain size 3 μm. ASTM F75 is a Co—Cr—Mo cast alloy that is currently actually used as a bone epiphysis material in artificial joints, and contains large amounts of carbides in order to improve the wear resistance. In the present example, it is indicated that the crystal grain size of the alloy prepared using high-temperature forging is greatly reduced.

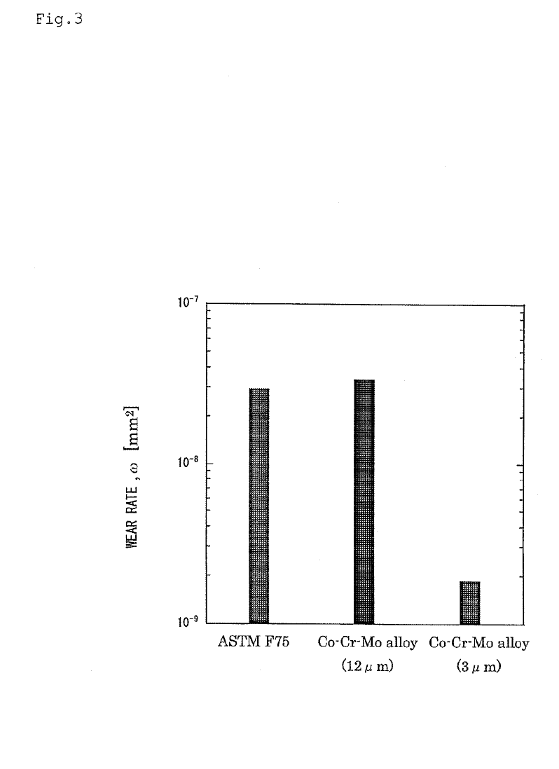

FIG. 3 shows the wear test results for ASTM F75 and Co-29Cr-6Mo alloys (with a finely reduced crystal grain size) prepared using high-temperature forging. It is indicated that compared to the wear rate of ASTM F75, the prepared Co—Cr—Mo alloy with a mean crystal grain size of 14 μm has about the same wear rate, and that the Co—Cr—Mo alloy with a mean crystal grain size of 3 μm has a far lower wear rate than these alloys. Furthermore, the high-temperature forging was performed as described above.

### Example 2

(Wear Test Results for Alloys With Increased Amount of Added Me)

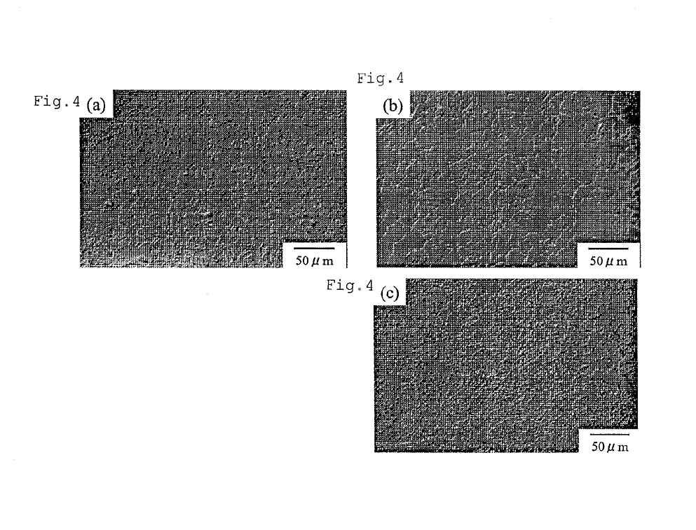

FIGS. 4(a), (b) and (c) show the optical-microscopic structures of Co-29Cr-xMO (x=6, 8, 10) forged alloys in which the respective amounts of Mo added were increased to 6, 8, and 10 mass %. In the 10Mo alloy, in which the crystal grain size was approximately 14 μm, but the amount of Mo added was large, fine deposition of the σ phase was recognized. Forging was performed by high-temperature forging in the same manner as in Example 1

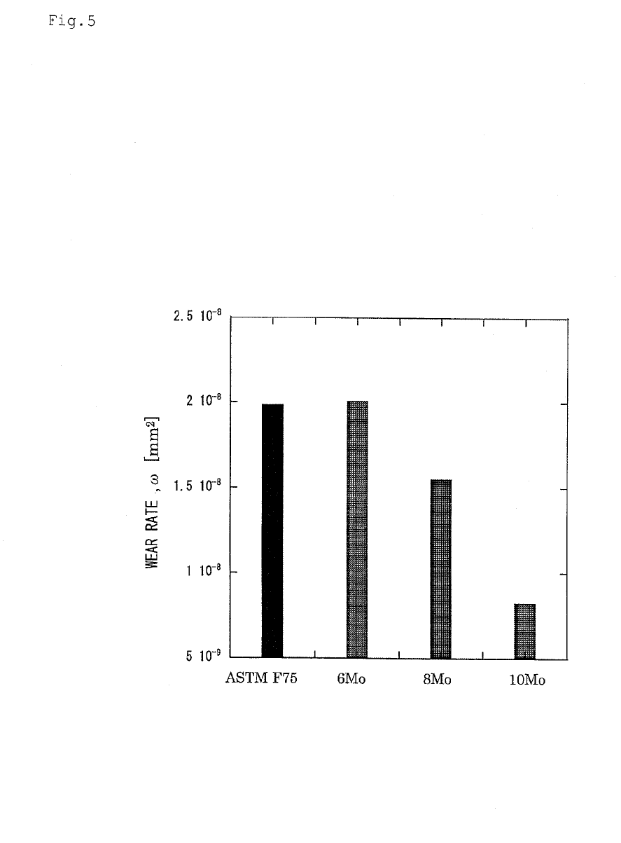

FIG. 5 shows the wear test results for Co-29Cr-xMo (x=6, 8, 10) forged alloys in which the amount of Mo added was increased to 6, 8, and 10 mass %, and for ASTM F75. The wear rate of the 6Mo alloy (Co-29Cr-6Mo alloy) showed no great difference from that of ASTM F75; however, the wear rates of the 8Mo alloy (Co-29Cr-8Mo alloy) and 10Mo alloy (Co-29Cr-10Mo alloy) were lower than that of ASTM F75. This indicates that the wear rate of Co—Cr—Mo alloys is reduced as the amount of Mo added is increased. This is thought to be due to the fact that in addition to a reduction in the crystal grain size, the wear resistance characteristics are improved as a result of the fine deposition of the σ phase as the amount of Mo is increased. As a result, it may be said that the fine deposition of the σ phase is also effective in improving the wear resistance characteristics of materials of the same type at the lubrication boundaries.

It is seen that if an increase in the amount of Mo added and a reduction in the alloy crystal grain size by high-temperature forging are combined, the σ phase is finely deposited (the dispersed deposition of the σ phase is reinforced), and an effect is obtained which improves the wear resistance characteristics.

### Example 3

(Wear Resistance Test of Sinters of Co-29Cr-6Mo Alloy Powders Prepared by Gas Atomization Method)

(Test Method)

A Co-29Cr-6Mo cast material (600 g) prepared using a vacuum induction melting furnace was used as the starting raw material. This was melted at a high frequency, and atomized in an Ar atmosphere. Sintering was performed using a vacuum high-temperature sintering furnace (hot press: manufactured by NEMS) using the alloy powder thus prepared (grain size 25 μm or less). Sintering was performed at 936° C. and 1052° C., at a pressing pressure of 40 MPa.

(Results)

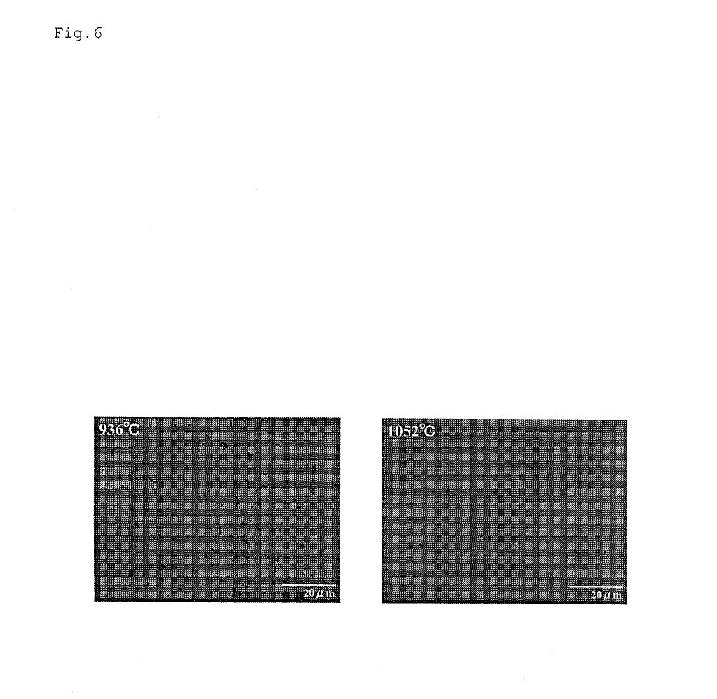

The optical-microscopic structures of the 936° C. and 1052° C. sinters are respectively shown in FIGS. 6A and 6B. It was found from an X-ray diffraction test that in the structure of the 936° C. sinter, an HCP phase was the main constituent phase, but that a σ phase was also included to a slight extent. The porosity in this case was 1 to 10%. Furthermore, it was found that the structure of the 1052° C. sinter was an FCC single-phase structure. The porosity in this case was 1 to 10%.

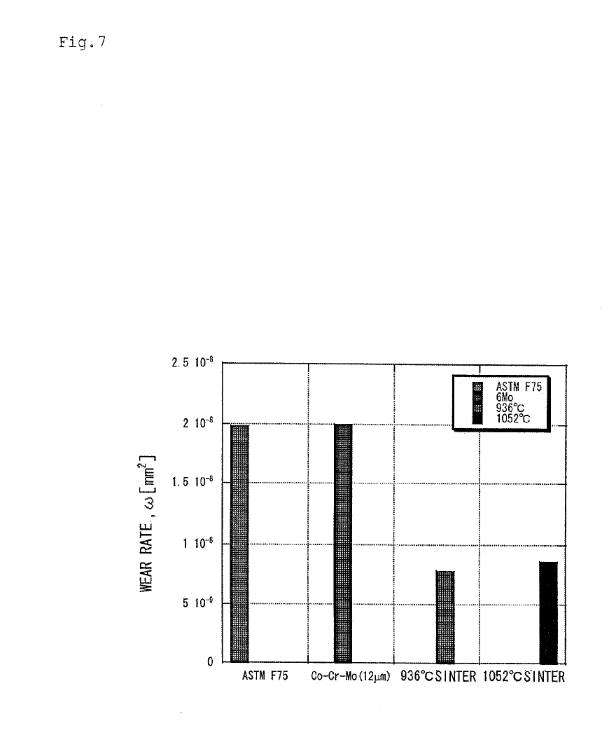

FIG. 7 shows the wear test results for the 936° C. sinter and the 1052° C. sinter. For purposes of comparison, the wear test results for ASTM F75 and a Co-29Cr-6Mo forged material (crystal grain size 12 μm) are also shown. It was found from these results that both the 936° C. and the 1052° C. sinter showed wear resistance characteristics surpassing those of the forged material. It is thought that this is due to the fact that a lubricating liquid collecting effect is shown by the pores appropriately included in the surface of the test material, in addition to the reduction of the crystal grain size, and a lubricating effect is obtained which is higher than that of the forged material in which no pores are present.

(Summarization of Wear Test)

The reduction in the crystal grain size of the Co—Cr—Mo alloy and the increase in the amount of added Mo improve the wear resistance in materials of the same type. The fine deposition of a σ phase in a fine crystal grain structure has an extremely high effect in improving the wear resistance in materials of the same type. Moreover, it was found that the utilization of pores in sinters prepared by powder sintering or the like has a high lubricating effect, and improves the wear resistance characteristics of materials of the same type.

In the present invention, the wear resistance characteristics of Co—Cr—Mo alloys for use in artificial joints can be improved by reducing the crystal grain size to a fine grain size, and the generation of wear debris in the living body can be suppressed (an amount of wear that is 1/10 that seen in a conventional product can be realized). For example, it was made clear that the wear resistance characteristics can be greatly improved even if no carbides are included, by reducing the crystal grain size to approximately 20 μm. Moreover, in the case of alloys in which the wear resistance characteristics were thus improved by reducing the crystal grain size to a fine grain size, when a wear test was performed in a quasi-biological liquid, it was made clear that the wear resistance characteristics were greatly improved compared to those of a conventional ASTM standard F75 alloy of the same type containing carbides. This result was obtained because of the absence of carbides attacking the other materials.

Furthermore, as a result of performing a wear test in a quasi-biological liquid in similar materials of the same type using alloys containing more Mo than a Co-29Cr-6Mo alloy, i.e., Co-29Cr-8Mo and Co-29Cr-10Mo alloys, in which the crystal grain size was similarly reduced to a fine grain size by high-temperature forging, it was made clear that wear resistance characteristics better than those of conventional materials were shown. It is thought that is due to the manifestation of an effect whereby the hardness of the overall alloy is increased by a σ phase that is deposited in a dispersed manner. Thus, the alloy of the present invention can be used in medical devices such as artificial hip joints, artificial knee joints, and the like which have a low toxicity in the living body, i.e., which are safer, and which have a long service life.

### INDUSTRIAL APPLICABILITY

The present invention can provide a Co—Cr—Mo alloy for use in artificial joints which is superior in terms of wear resistance characteristics, a method for manufacturing same, and materials for use in the body and artificial replacement materials which can be manufactured from this alloy. The present invention can provide a technique in which the wear resistance characteristics of a Co—Cr—Mo alloy for use in artificial joints is improved, and the generation of wear debris in the living body is suppressed, using a simple and inexpensive method. Accordingly, the alloys that are obtained are superior in terms of cost, and can be used in a wide range of practical applications, e.g., to manufacture biocompatible materials and medical devices.

It is clear that the present invention can also be implemented in applications other than the description and examples given above. In accordance with the abovementioned teaching, many alterations and modifications of the present invention are possible. Accordingly, these are also included in the scope of the claims appended to the present application.

1. A method for improving wear resistance characteristics in a Co—Cr—Mo alloy for use in artificial joints, characterized in that a treatment selected from the following group is performed: (1) a treatment for reducing the crystal grain size of the alloy, (2) preparation of the alloy composition in which the Mo content is enriched, (3) a treatment for reinforcing the dispersed deposition of the σ phase, (4) a powder sinter formation treatment, and (5) a treatment for forming pores generated in the powder sinter.

2. The method for improving the wear resistance characteristics of a Co—Cr—Mo alloy according to claim 1, characterized in that the Co—Cr—Mo cast alloy is subjected to a high-temperature forging treatment, and the crystal grain size of the alloy is reduced to a fine grain size.

3. The method for improving the wear resistance characteristics of a Co—Cr—Mo alloy according to claim 1, characterized in that the mean grain size of the alloy crystals is set at a grain size selected from the group consisting of (1) 20 μm or less, (2) 15 μm or less, (3) 13 μm or less, (4) 11 μm or less, (5) 9 μm or less, (6) 7 μm or less, (7) 5 μm or less, (8) 4 μm or less, (9) 3.5 μm or less, (10) 3 μm or less, (11) 2.5 μm or less, (12) 2 μm or less, (13) 1.5 μm or less, and (14) 1 μm or less.

4. The method for improving the wear resistance characteristics of a Co—Cr—Mo alloy according to claim 1, characterized in that the Mo content in the alloy is set at an amount selected from the group consisting of 1) 6 mass % or greater, (2) 6.5 mass % or greater, (3) 7 mass % or greater, (4) 7.5 mass % or greater, (5) 8 mass % or greater, (6) 8.5 mass % or greater, (7) 9 mass % or greater, (8) 9.5 mass % or greater, (9) 10 mass % or greater, (10) 11 mass % or greater, and (11) 12 mass % or greater.

5. The method for improving the wear resistance characteristics of a Co—Cr—Mo alloy according to claim 1, characterized in that an alloy composition is used in which the Mo content is enriched, and the dispersed deposition of the σ phase is reinforced by performing a high-temperature forging treatment.

6. The method for improving the wear resistance characteristics of a Co—Cr—Mo alloy according to claim 1, characterized in that the alloy is an alloy in which the amount of elements other than the elements Co, Cr, and Mo present in the alloy composition is 1 mass % or less.

7. The method for improving the wear resistance characteristics of a Co—Cr—Mo alloy according to claim 1, characterized in that the cast alloy is subjected to a gas atomization treatment, the resulting alloy powder is sintered, and pores are formed in the surface of a member.

8. The method for improving the wear resistance characteristics of a Co—Cr—Mo alloy according to claim 1, characterized in that the sintering of the alloy powder is performed at a temperature selected from the group consisting of (1) 600° C. to 1350° C., (2) 650° C. to 1300° C., (3) 700° C. to 1250° C., (4) 750° C. to 1200° C., (5) 800° C. to 1150° C., (6) 850° C. to 1100° C., (7) 875° C. to 1060° C., and (8) 900° C. to 1050° C.

9. The method for improving the wear resistance characteristics of a Co—Cr—Mo alloy according to claim 1, characterized in that that the sintering of the alloy powder is performed at a pressure selected from the group consisting of (1) 10 to 250 MPa,(2) 20 to 200 MPa, (3) 25 to 150 MPa, (4) 30 to 150 MPa, (5) 30 to 100 MPa, (6) 30 to 80 MPa, (7) 35 to 50 MPa, (8) 35 to 45 MPa, and (9) 10 to 60 MPa.

10. A Co—Cr—Mo alloy for use in artificial joints in which the wear resistance characteristics are improved, characterized in that this alloy is treated by a treatment selected from the group consisting of treatments in which (1) the crystal grain size of the alloy is reduced to a fine grain size, (2) the alloy has a composition in which the Mo content is enriched, (3) the dispersed deposition of the σ phase is reinforced, (4) a powder sinter formation treatment is performed, and (5) pores are formed in the powder sinter.

11. The Co—Cr—Mo alloy for use in artificial joints accordingly to claim 10, characterized in that this alloy is manufactured using the method for improving wear resistance characteristics according to claim 1.

12. A medical device characterized in being manufactured from the Co-Cr-Mo alloy according to claim 10.

13. The medical device according to claim 12, characterized in being an artificial joint.

14. The medical device according to claim 13, characterized in being a bone epiphysis and acetabular roof for use in artificial joints.
